(19) United States
(12) Patent Application Publication
(10) Pub. No.: US 2009/0014001 A1
(43) Pub. Date: Jan. 15, 2009

(54) METHOD AND APPARATUS FOR PROVIDING VENTILATION AND PERFUSION

(76) Inventors: Helge Myklebust, Stavanger (NO); Joar Eilevstjonn, Sandnes (NO); Jon Nysaether, Hafrsfjord (NO)

Correspondence Address: Karen Henckel, Esq.;DORSEY & WHITNEY LLP, Suite 3400, 1420 Fifth Avenue, Seattle, WA, 98101, US

(21) Appl. No.: 12/163,955

(22) Filed: Jun. 27, 2008

(30) Foreign Application Priority Data
Jun. 29, 2007 (GB) 0712710.3

(51) Int. Cl. A61M 16/00; A61B 5/00

(52) U.S. Cl. 128/204.18; 128/205.18; 128/205.24; 600/301

(57) ABSTRACT

The present invention relates to a device and method for providing ventilation and perfusion in a patient. The device comprises an airway pressure supply device and a controller for controlling the airway pressure of the airway pressure supply device. In one embodiment, the airway pressure supply device comprises a piston moveably arranged in a cylinder and a motor connected to the piston. Activation of the airway pressure supply device is performed by moving the piston in the cylinder by the motor.

### CROSS-REFERENCE TO RELATED APPLICATION(S)

This application claims the benefit of United Kingdom Application No. 0712710.3, filed Jun. 29, 2007. The entire disclosure of the prior application is considered to be part of the disclosure of the instant application and is hereby incorporated by reference therein.

### TECHNICAL FIELD

This invention relates to a device and method for delivering ventilation and perfusion to the airways of a patient.

### BACKGROUND OF THE INVENTION

Despite investments in research, training, equipment and infrastructure, the survival rate from an unexpected cardiac arrest has been virtually unchanged over the past couple of decades. On average, the survival rate is 5-10% in the United States and Europe, but can be as low as 2% in larger cities or well over 20% in cities with the best implementation of science and education.

One factor influencing survival rates is time elapsed from cardiac arrest onset until professional treatment begins. This time may vary greatly. It is known that the vital organs can sustain approximately 5-10 minutes without perfusion. However, after this time interval, cell death increases and irreversible organ damage begins. There is, thus, a need for expanding the time window of opportunity that one can recover from cardiac arrest. Cardiopulmonary resuscitation (CPR) is the most common method to generate perfusion in the arrested victim, and CPR is known to slow down the process of cell death.

A factor that influences the chances of a victim's survival is that current treatment (i.e., chest compressions, ventilations, defibrillation, and drugs) does not address the underlying cause of the arrest. Furthermore, some hearts are simply too compromised to be restarted, even though CPR, drugs, and defibrillation are delivered according to best practices. Many patients who do not receive a return of spontaneous circulation within some minutes of resuscitation attempts could benefit from receiving continuous CPR to keep vital organs intact, followed by application of some external means of circulation to buy enough time so that corrective treatment can be done in the hospital.

Another factor affecting survival is reperfusion injury. Cell death does not only takes place as a result of ischemia, but also as a function of reperfusion. Given the situation of sudden cardiac arrest, there has been evidence to suggest that most of the cell death and subsequent irreversible organ damage takes place when perfusion is restored. This is due to the circulation of toxic components that have built up during ischemia. This is described in further detail by Vanden Hoek, et. al. “Reperfusion, Not Simulated Ischemia, Initiates Intrinsic Apoptiosis Injury In Chick Cardiomyocytes,” Am J Physiol Heart Circ Physiol, 284:H141-H150, 2003.

A third factor that can improve survival rates is induced hypothermia. It is known that therapeutic hypothermia is beneficial after cardiac arrest. Furthermore, it is known that intra-arrest cooling is beneficial with respect to both defibrillation success and survival after discharge from the hospital. Cooling appears to slow down the speed of cell death caused by reperfusion after cardiac arrest. This is further described by Abella, et al. in “Intra-Arrest Cooling Improves Outcomes in a Murine Cardiac Arrest Model,” Circulation 2004; 109; 2786-2791.

As already mentioned, CPR is the most used method to prevent cell death and organ injury. The method comprises chest compressions and ventilation, sometimes interrupted for defibrillation. There are, however, limits to this method. The person performing CPR may not be sufficiently skilled or motivated, and even if skilled, many rescuers do not perform CPR very well. (JAMA, January 2005, Wik et al., Abella et al.). Additionally, there are difficulties associated with performing CPR in an ambulance and often there are not enough rescuers available to perform CPR and perform other necessary activities at the same time. Furthermore, it is difficult to perform CPR over a long period of time and the effectiveness of CPR to generate flow is reduced by time as vascular tone decreases. These limitations have led to the development of devices that can substitute the manual delivery of CPR and/or enhance the effectiveness of delivered CPR.

U.S. Pat. No. 4,397,306 describes an integrated system for cardiopulmonary resuscitation and circulation support. The integrated system comprises a chest compression machine which compresses the patient's sternum at desired intervals and to a desired degree. It includes a lung ventilation means, which includes a high pressure ventilator for ventilating simultaneously with chest compression, a low pressure ventilator for inflating the lungs at low pressure between a selected number of compression cycles, and a negative pressure ventilator for deflating the lungs between chest compressions. The system further comprises a means for restricting the abdomen to exert pressure on the abdominal wall and a control means for selectively operating the chest compression means, the lung ventilating means, the valve means and the abdomen restriction means. This is a very complex system and the effect is based on the interaction and cooperation of the plurality of units of the system, particularly, the coordination between the ventilation of the lungs and the chest compressions. In this system, perfusion is mainly caused by chest compressions and is somewhat enhanced by using alternating positive and negative pressure connected to the lungs and a vest.

The impedance threshold valve (ITV) is another device that uses a combination of chest compressions and controlled ventilations. A general description of the ITV device and principle as applied to cardiac arrests is to provide a small negative pressure in the airways in between chest compressions so that venous return is enhanced. A positive airway pressure is generated as a result of positive pressure ventilation using a separate device, such as a self-inflating bag and mechanical ventilator. However, because the magnitude and timing of negative pressure versus positive pressure is not controlled or optimized, the resulting effect might be that the negative pressure causes accumulation of fluid in the lungs as well as atelectatis (lung collapse) with pulmonary blood shunting and lung oedema.

U.S. Pat. No. 5,551,420 describes one arrangement of the ITV in which chest compressions and decompressions are used to generate perfusion. The ITV is used to enhance perfusion by preventing some return of air to the lungs as the chest recoils between compressions, and thereby generating a small but controlled level of negative airway pressure between compressions. According to the patent disclosure, negative pressure can be created either by valve arrangement or by increasing the impedance of the gas returning to the patient's lungs.

U.S. Pat. No. 5,692,498 is a further embodiment of the ITV where either a facial mask or a ventilation tube is arranged with a pressure-responsive valve system set to generate and control a small level of negative airway pressure between compressions. Systems which combine controlled ventilation and negative airway pressure with chest compressions are further described in, for example, U.S. Pat. No. 6,062,219, U.S. Pat. No. 5,692,498, U.S. Pat. No. 6,604,523, U.S. Pat. No. 6,526,973, and U.S. Pat. No. 5,551,420. Common to these inventions is the use of chest compressions as the main source of perfusion.

Many more patents or applications describe methods and/or systems for treating victims. U.S. Pat. No. 6,155,257 is yet another embodiment of the ITV principle, where a sensor is provided to detect chest compressions and a controller arranged to control actuation of a ventilator to deliver positive and negative pressures of air to the lungs.

U.S. Pat. No. 6,938,618 is about a method of treating cardiac arrest, where chest compressions are the main source of perfusion, and where negative and positive airway pressures are delivered to further enhance perfusion. Also, for patients who suffer low blood pressure or head trauma, but who are not in cardiac arrest, the method of using positive and negative airway pressures is disclosed. A self inflating bag is described as the means of providing positive and negative airway pressures.

U.S. Pat. No. 7,082,945 discloses a method for treating victims of head trauma by lowering intra-cranial pressures by delivery of positive and negative airway pressures. A selection of devices is suggested, including a thoracic vest, ventilator bag and mechanical ventilator. Again, in this invention, the patient is not in cardiac arrest and the invention is not directed to treat cardiac arrest by causing sufficient levels of perfusion.

U.S. Pat. No. 6,986,349 and U.S. Pat. No. 7,210,480 both describe using the ITV principle to enhance perfusion in patients who are breathing spontaneously. Also in these inventions, the patient is not in cardiac arrest and the invention is not directed to treat cardiac arrest by causing sufficient levels of perfusion.

U.S. Pat. No. 7,195,012 describe a method of treatment for patients in need of reduced intracranial pressure by the application of the ITV principle. Again, in this invention, the patient is not in cardiac arrest and the invention is not directed to treat cardiac arrest by causing sufficient levels of perfusion.

U.S. Patent Publication No. 2003-0062040 describes a face mask ventilation/perfusion system, where a pressure sensitive valve is arranged according to the ITV principle. U.S. Patent Publication No. 2003-0037784 describes using the ITV principle to enhance blood circulation in breathing persons.

U.S. Patent Publication No. 2005-0217677 discloses a bag-valve device that takes advantage of the ITV principles in order to enhance blood flow while CPR is delivered to victims of cardiac arrest or to enhance venous return in patients suffering low blood pressure and head trauma.

U.S. Patent Publication. 2004-0231664 discloses a ventilator for treating head trauma and low blood circulation. A valve system is connected to the patient's airway to reduce intrathoracic pressures during each inspiration. Further connected to the valve system is a vacuum source and regulator. A nerve stimulator might be used to further enhance the negative intrathoracic pressure.

Another way to provide some circulation is to make the patient cough. Coughing changes pressure in the lungs, which force air in and out of the airways and the lungs. There are case reports (first from Jama 1976) on patients in cardiac arrest which have been able to sustain consciousness by vigorous coughing for as much as 90 seconds. (See C2005 COIs used as data supplement, Resuscitation 2005; 67). In this situation, the coughing muscles will first produce a negative airway pressure causing air to fill the lungs followed by a positive pressure gradient moving air out of the lungs. The same pressure gradients which cause air to move in and out of the lungs are also the driving force of blood that is sucked in from the venous side and delivered out on the aortic side.

There are also observations where positive pressure ventilation, itself, causes a small level of end-tidal carbon dioxide (EtCO₂) in animals in cardiac arrest, despite the heart having stopped (asystole). This can be explained by the positive airway pressure which forces blood out of the lungs and out of the thorax, and the return of blood to the venous side and lungs between ventilations, where the direction of the blood is controlled by the valves in the heart and in the veins. Furthermore, there are observations that the negative airway pressure caused by gasping under cardiac arrest improves cerebral perfusion pressure. (Circulation, volume 114, No 18, Oct. 31, 2006; ReSS abstracts 55 and 36.)

U.S. Pat. No. 6,415,791 describes an airway clearance system and method which produces cough-like forces on a patient in order to clear the airways of the patient. This system and method is suitable for patients with mucus problems due to diseases such as cystic fibrosis, emphysema, asthma, chronic obstructive pulmonary disease, and chronic bronchitis. The system comprises a belt which induces external forces on the chest/lungs of the patient and an air pressure supply connected to the patient's mouth. The air supplied to the patient's lungs through the mouth is pulsed with positive pressure pulses coordinated with the pressure/force pulses induced on the chest in order to simulate a cough and clear the upper airway passages from mucus. The purpose of the air pressure supply in this system is to help clear mucus from the upper airways of the patient, while the belt transports the mucus from the lower airways/lungs to the upper airways. Sequence of operation is first to provide a positive airway pressure that expands the chest followed by a rapid negative pressure gradient to help move air out of the lungs.

From manufacturer J.H. Emerson Co., the product CoughAssist® is currently on the market. This is a device which delivers alternating positive and negative pressure to the lungs for the purpose of clearing retained bronchopulmonary secretions.

U.S. Pat. No. 2,364,626, also by Emerson, discloses a resuscitator for the purpose of resuscitating victims of drowning, gas asphyxiation and the like. Compressed gas is the energy source, and valves and venturi principles are used to generate alternating positive and negative pressures. U.S. Pat. No. 2,468,741, by the same inventor, is a similar device now designed as a breathing apparatus for patients who are unable to breathe normally.

U.S. Pat. No. 5,345,930 discloses a method and apparatus for assisting the movement of pulmonary secretions via supramaximal flow. In this patent, a vacuum pump is connected to a vacuum cylinder, and a quick response valve connects the vacuum to a face mask in order to facilitate negative pressure to the airways such that respiratory gases with mucus are extracted with coughing.

Exsufflation with negative pressure (EWNP) and device is discussed in the paper “Physiological effects of E.W.N.P., Diseases of The Chest,” January 1956, Vol. 29, pp. 80-95. Specifically, this paper describes a breathing control procedure in which the positive pressure was built up to 40 mm Hg above atmospheric in two seconds, then a pressure drop to 40 mm Hg negative pressure would occur in 0.04 sec. The negative pressure was maintained for approximately 1.5 sec. Interestingly, using this device resulted in increased cardiac outputs, from just below 5 l/min to about 6.8 l/min, suggesting that the use of alternating positive and negative pressure is feasible for providing both perfusion and ventilation.

The prior art devices and methods described above use alternating positive and negative airway pressures to support breathing, provide coughing effects, enhance perfusion caused by chest compressions or for patients not in cardiac arrest, and/or cause favorable intracranial pressures for victims of traumatic brain injury.

Because, as described above, CPR itself has limited effect, there is a need for a simple device that provides sufficient ventilation and circulation under cardiac arrest, while being simple and cheap enough to be widely used by ambulance personnel, and compact and self contained to fit with the workflow.

### SUMMARY OF THE INVENTION

The present invention is directed toward a device and method for providing perfusion to airways of a patient. In one aspect of the invention, an apparatus includes an airway pressure supply device operable to provide positive and negative pressurized gas to the airways of the patient and a controller for controlling the pressure of the airway pressure supply device.

### BRIEF DESCRIPTION OF THE DRAWINGS

FIG. 1 is a schematic illustration of the airway pressurizing device according to one embodiment of the invention.

FIG. 2 is a block diagram of the airway pressurizing device connected to and operating with the body of a patient according to one embodiment of the invention.

### DETAILED DESCRIPTION OF PREFERRED EMBODIMENTS

Embodiments of the present invention are directed toward a device and method for providing perfusion to airways of a patient. Certain details are set forth below to provide a sufficient understanding of the embodiments of the invention. However, it will be clear to one skilled in the art that various embodiments of the invention may be practiced without these particular details.

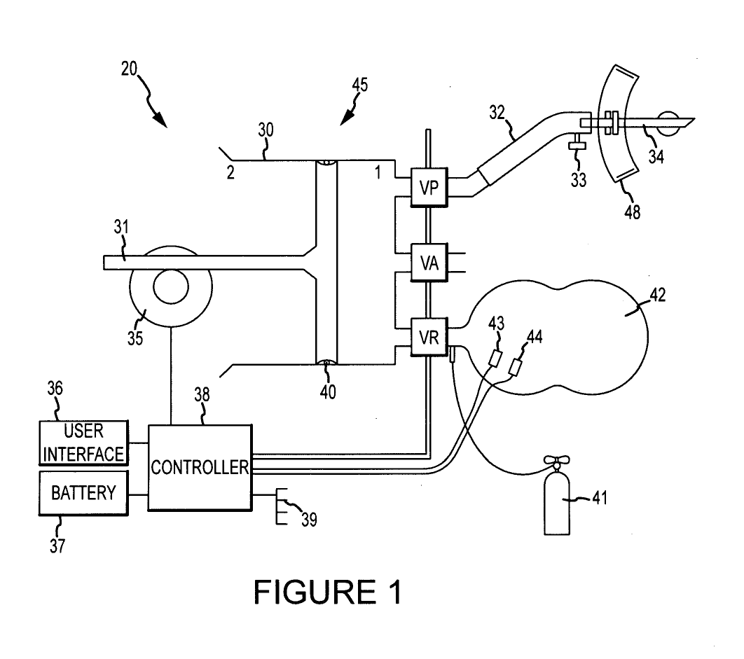

FIG. 1 is a schematic drawing of an airway pressurizing device 20 according to one embodiment of the invention. The airway pressurizing device 20 is a device for creating a flow of pressurized gas into the airways of a patient. The pressurized gas may be any gas, such as air, oxygen, a combination of oxygen and CO₂ or a combination of air and CO₂ or other gases or pulverized substances or a mixture of these. In the following, embodiments of the invention will be described by using air as an example. However, in practice, any of the above substances or other gases can be used.

The airway pressurizing device 20 comprises an airway pressure supply device 45 and a controller 38. The controller 38 controls the airway pressure supply device 45, and thus airway pressure. Variables that may be controlled by the controller 38 include magnitude and/or sign of the airway pressure and timing of pressure changes by setting frequency and duration of pressure pulses. The controller 38 may comprise a processor and may be connected to a user interface and/or sensors. When connected to sensors or a user interface, the controller may receive input for processing and may be able to process and evaluate input data and change the airway pressure accordingly.

In the embodiment in FIG. 1, the airway pressure supply device 45 comprises a piston 31 moveably arranged in a cylinder 30, and a motor 35 coupled to the piston 31 and to the controller 38. In one embodiment, the motor 35 is a servo motor, enabling rapid acceleration and de-acceleration. In some embodiments, the power source 37 for the motor 35 may be a battery, such as a battery from manufacturer A123, preferably using a stack of ANR26650M1 cell giving in excess of 30 minutes of operation time. The controller 38 is powered by the power source 37. As stated above, the controller may comprise one or more microprocessors. In the illustrated embodiment, the controller 38 is coupled to sensors 33, 39, 43, 44, valves VR, VA, VP, and motor 35. As will be explained below, the controller 38 can control the operation of the valves VR, VA, VP in order to control the pressure provided by the airway pressure supply device 45. A user interface 36 is coupled to the controller 38. In some embodiments, the user interface 36 may have input devices such as switches and dials, and output devices such as displays and alarms.

As mentioned above, the pressure supply device 45 comprises a piston, which in the illustrated embodiment is a large bore piston 31 operated by the electric motor 35. Piston 31 is situated moveably within cylinder 30. In some embodiments, the volume of cylinder is about 6-10 liters, however, other volumes may be used. Preferably, the edges of the piston 31 are sealed by means of a suitable sealing 40 towards the walls of the cylinder 30.

A gas reservoir 42 is connected to the cylinder 30 bottom through the valve VR. The reservoir 42 contains air to be used during resuscitation. One or more sensors may be arranged in the reservoir 42 to provide feedback regarding the gas composition in the reservoir 42. In FIG. 1, a CO₂ sensor 43 and an O₂ sensor 44 are arranged within the reservoir 42 to measure the gas composition. An exhaust valve VA is also connected to the cylinder 30 to allow connection to ambient pressure. A gas tank 41 may be removably coupled to the gas reservoir and capable of filling the gas reservoir with a gas. In some embodiments, the filling rate may be 5-20 liters/minute.

A patient interface 32 is coupled to the cylinder 30 through valve VP to deliver gases to the patient and to receive gases from the patient. In one embodiment, the patient interface 32 is a flexible yet non-compressible tubing that can sustain both positive and negative pressures. The patient interface 32 may include a device for providing a secure airway to the patient. This may be a tube, combitute, laryngeal mask or other suitable methods for enabling transport of air in and out of the airways without substantial leakage. In reference to FIG. 1, a patient tube 34 is connected to the patient interface 32. In one embodiment, the tube is of the endotracheal type. In some embodiments, a tube holder 48 is connected to the tube 34 to restrict movement of the tube 34 as gas is moved rapidly in and out of the patient. For example, in one embodiment the tube holder 48 is a Thomas™ Tube Holder.

In other embodiments, the patient interface 32 includes a pressure sensor 33 to set and/or measure airway pressures, i.e., the gas pressure in the airway of the patient. The pressure sensor 33 may be arranged in the secured airway as an integrated part of the device for providing a secure airway, or can be arranged as a separate unit. The output from the pressure sensor 33 may be used by the controller 38 to regulate the pressure from the airway pressure supply device 45 and obtain an optimal airway pressure. The sensor 33 may be coupled to the controller 38 as feedback on the effectiveness of the pressure pulses and can be used by the controller 38 to optimize the magnitude and duration of the airway pressurization as a function of the measured effectiveness.

The function of the airway pressure supply device 45 is best described by describing an example of a sequence of stages. In the first stage, with valves VA and VR closed and valve VP open, the piston 31 starting from position 1 (right side of cylinder 30 in FIG. 1) starts to move to position 2 (left side of cylinder 30 in FIG. 1) building up a negative pressure in the cylinder 30 and in the patient airways until a desired negative pressure is reached. The pressure may be monitored by pressure sensor 33, in which the pressure measurements may be fed to controller 48. The time needed to build the desired negative pressure is patient dependant, and is recorded by the controller and recorded as time period T1.

In the second stage, valve VP first closes and valve VR opens. This causes the negative pressure within the patient airways to be maintained for a controlled time period T2, during which the piston 31 continues to move to position 2 now extracting gas from the reservoir in preparation for the next stage. During the first and second stage, the patient airway pressure will be negative. This results in increased return of venous blood towards the right side of the heart as well as filling of pulmonary vascular capacitance with blood.

In the third stage, VR closes and now with valves VR and VA closed, VP opens as the piston 31 begins to move towards position 1 and thereby pressurizes the airways. Movement towards position 1 continues until a desired positive airway pressure has been reached and positive pressure is maintained for a time period T3, set by the controller. This now results in movement of blood from the pulmonary vascular capacitance to the left side of the heart and further out to the aorta. Because of the pulmonary valve as well as the aortic valve, blood is circulated in the forward direction.

In the fourth stage, having a duration T4, with the piston 31 not moving, valves VP and VR may be opened to allow some or most of the compressed gas to be re-directed back to the patient reservoir 42. Alternatively, or in combination, valve VA opens to let some or most of the compressed gas out to ambient. T4 is recorded by the controller 38 as the time needed for the patient airway pressure to reduce to about ambient pressure level. In a brief, fifth stage, with valves VR and VP closed, and with VA open, the piston is brought quickly to the position 1 in preparation for repeating the first stage.

As will be appreciated by those skilled in the art, the airway pressure supply device 45 may be any kind of device which is capable of performing the above mentioned operation, for example, compressed gas containers, pumps, piston and cylinder, compressors, etc. Additionally, the airway pressure supply device 45 may be used with or without other means for exerting external forces on the chest or lungs of the patient. For instance, the airway supply pressure device 45 can additionally include or be used in combination with devices for abdominal and/or chest binding. Such devices may brace the chest/abdomen and reduce lung compliance, thereby improving the pressure gradients. In one embodiment, a set of belts may be arranged to limit the movement of the patient's chest and belly, and thereby enhance the pressure gradients. This would also direct more of the flow to the vital organs, such as the brain and heart.

In preparation for use, the patient may first be intubated, and the tube secured as normal, while traditional, manual CPR is delivered. Then the tube will be connected to the apparatus using the patient interface. The gas reservoir is connected to valve and outlet VR, and filled with a desired gas.

In another embodiment, the airway pressure supply device comprises two units, one positive pressure unit and one negative pressure unit. The positive pressure unit may be arranged as a compressor/pump connected between a positive pressure reservoir and a negative pressure reservoir, where a valve is arranged to connect either the positive reservoir or the negative reservoir through a dedicated regulator to the secured airway. Pressurized gas may be used as the source of the positive pressure and a vacuum reservoir as the source of the negative pressure. The regulator and/or valve is connected to the controller to be able to control the magnitude and duration of short or longer pressure pulses or a constant airway pressure.

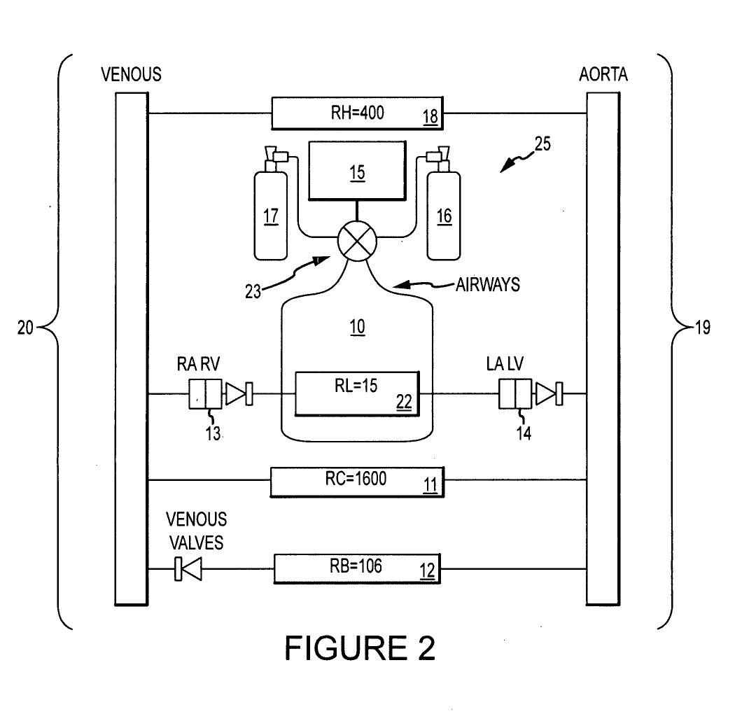

FIG. 2 is a block diagram of an airway pressurizing device 25 being used in conjunction with a vascular system of a patient according to another embodiment of the invention. The vascular system comprises left and right sides of a heart (not shown) on either side of a lung tissue 22. In FIG. 2, RA denotes Right Atrium, RV denotes Right Ventricle, LA denotes Left Atrium, and LV denotes Left Ventricle. The numbers in the figure are typical units of vascular resistance associated with the different portions of the body. From this it can be seen that the vascular resistance across the lungs is relatively low. For a typical flow of 5 l/min, average perfusion pressure is about 15 mmHg, and 15 mmHg is about 20% of the typical perfusion pressure needed to circulate the large circulatory system. Typically, the lung tissue 22 can hold between 0.5 and 1.0 litres of blood (pulmonary vascular capacitance), and a typical systemic blood flow is 5/min. However, it is known that cardiac arrest and states of shock lead to reduced vascular tone and hence reduced vascular resistance. With reduced vascular resistance, less perfusion pressure is needed to generate sufficient circulation.

Airways 10 of the patient are indicated as a bottle shaped volume comprising the lung tissue 22. A pulmonary valve 13 and an aortic valve 14 determine the direction of flow through the lung tissue 22. A regulator and valve 23 is connected between the airways 10 and a positive pressure reservoir and pump 17 and a negative pressure and reservoir pump 16. A controller 15 is connected to the regulator and valve 23 and controls the regulator and valve. In this configuration, a selected specific magnitude of negative or positive airway pressure can be provided for a selected time period. In one embodiment there are provided a sequence of succeeding positive and negative airway pressure pulses or periods. In one modified embodiment, one pump (not shown) is arranged between the positive and negative pressure reservoir, working to keep the pressures in each reservoir within desired values.

When negative pressure is applied by means of the negative pressure reservoir and pump 16, blood will flow from the venous system 20 through the right side of the heart to accumulate in the lung tissue 22. When a positive pressure is applied by means of the positive pressure reservoir and pump 17, the accumulated blood will be forced out of the lung tissue 22, filling the right side of the heart and even filling the aorta 19 while the aortic pressure is below the applied positive pressure. From the aortic side 19, blood flow is then distributed through the different organs (head and arms 18, coronary 11, thorax, abdomen and lower extremities 12) as a function of vascular resistance, as indicated by the numbers in FIG. 2.

As an indication of typical magnitudes of airway pressure, a small scale experiment involving three volunteers was performed:

Results:

| Typical positive airway pressure with coughing | 75 to 150 | cmH₂0 |
|---|---|---|
| Typical negative airway pressures with coughing | −50 to −100 | cmH₂O |
| Typical cough volumes: | 1-2 | liters |
| Typical total effective lung volumes | 3.5-7 | liters |

| | |
|---|---|
| Cough volume of total effective lung volume | 20-30% |

According to these results, typically the magnitude of the negative airway pressure might be set to about −50 cmH₂O and the positive airway pressure might be set to about 100 cmH₂O. From ILCOR, International Liason Committee on Resuscitation's document “C2005 COI used as data supplement” published in Resuscitation 2005, volume 67, references to reports can be found where patients in the cath lab were able to maintain consciousness and a mean arterial pressure above 100 mmHg for up to 90 seconds while coughing once every 1 to 3 seconds. Based on this, the controller should be able to set the interval between applied negative airway pressures from about 1 second and up to about 10 seconds depending on the vascular resistance.

The selection of magnitudes for positive airway pressures and negative airway pressures is preferably empirically selected, as are the time periods T2, T3 and T5. However, in an alternative embodiment, biosensors which can indicate the effectiveness of the device can be used to optimize pressures and time intervals. Such other biosensors include, but are not limited to, oxygen saturation sensor, end tidal CO₂ sensor, invasive CO₂ sensor, skin color sensor, blood pressure sensor, blood flow sensor, or a combination of these. For perfusion of the brain, in particular, the measurement of brainstem auditory evoked response BAER is relevant. This technique, as described by Reid and co-workers in Resuscitation volume 36, 1998, uses series of auditory stimuli followed by series of measurement of the electrical potential generated by the brain. Two electrodes were used to measure the brain potential. The researchers found that cardiac arrest resulted in loss of BAER within 30s of arrest, and restoration of BAER within 1-2 minutes of CPR initiation. Hence, BAER signal response can be used to determine the effectiveness of the treatment with respect to brain perfusion.

In order to prevent cerebral vasoconstriction, it is necessary to control saturation of CO₂ in the blood. With hyperventilation, CO₂ is washed out from the blood, which causes cerebral vasoconstriction and subsequent poor tissue oxygenation. To prevent this, the gas which is used to pressurize the lungs should contain CO₂. As expired air contains about 4-5% CO₂, and expired air is used for mouth to mouth ventilation, it is feasible to use the patient's expired air as basis for pressuring the airway. This is easily achieved using the cylinder and piston embodiment as explained above. As an alternative, the above mentioned gas tank 41 might contain a mixture of 5% CO₂ and either 95% air or 95% oxygen.

As stated above, even though the airway supply pressure device has been described as a stand alone unit, it can be used in combination with other devices or techniques for providing even better perfusion/circulation. Such devices or techniques include, but are not limited to, manually performed external chest compressions, automated chest compressions, elevation of the lower body, etc.

Although the present invention has been described with reference to the disclosed embodiments, persons skilled in the art will recognize that changes may be made in form and detail without departing from the spirit and scope of the invention. Such modifications are well within the skill of those ordinarily skilled in the art. Accordingly, the invention is not limited except as by the appended claims.

What is claimed is:

1. An apparatus for providing ventilation and perfusion to airways of a patient, comprising:
an airway pressure supply device operable to provide a first pressurized gas and a second pressurized gas to the airways of the patient; and
a controller for controlling the pressure of the airway pressure supply device.

2. The device according to claim 1, wherein the airway pressure supply device comprises a positive pressurized reservoir operable to provide the first pressurized gas to the airways of the patient and a negative pressurized reservoir operable to provide the second pressurized gas to the airways of the patient.

3. The device according to claim 2 further comprising a valve operable to selectively couple the positive pressurized reservoir to the airways of the patient.

4. The device according to claim 1, wherein the airway pressure supply device comprises a piston moveably coupled to a cylinder and a motor coupled to the piston and the controller.

5. The device according to claim 4, wherein the airway pressure supply device comprises at least one valve.

6. The device according to claim 5 further comprising a gas reservoir coupled to the cylinder by the at least one valve.

7. The device according to claim 1 further comprising at least one sensor coupled to the controller.

8. The device according to claim 7, wherein the at least one sensor is one of an oxygen saturation sensor, an end tidal CO₂ sensor, a skin color sensor, a blood pressure sensor, and a blood flow sensor.

9. The device according to claim 1 further comprising a patient interface coupled to the airway pressure supply device, the patient interface providing a secure path to the airways of the patient.

10. The device according to claim 9, wherein the patient interface is one of a tube, combitube, and laryngeal mask.

11. The device according to claim 10, the patient interface comprises a pressure sensor or a transducer coupled to the controller operable to set or measure the pressure in the patient interface.

12. The device according to claim 1, further comprising belts operable to limit the movement of the patient's chest and belly.

13. An apparatus for providing ventilation and perfusion to airways of a patient, comprising:
a piston moveably engaged with a cylinder, the piston and cylinder being operable to alternatingly provide a first pressurized gas to the airways of the patient and a second pressurized gas to the airways of the patient; and
a controller coupled to the piston and operable to control movement of the piston relative to the cylinder.

14. The apparatus of claim 13 further comprising a valve coupling the cylinder to a reservoir, the reservoir comprising a third pressurized gas.

15. The apparatus of claim 13 further comprising a patient interface for coupling the cylinder to the airways of the patient.

16. An apparatus for providing ventilation and perfusion to airways of a patient, comprising:
a controller;
a first pressurized unit coupled to the controller and comprising a first pressurized gas, the first pressurized unit operable to selectively provide at least some of the first pressurized gas to the airways of the patient; and
a second pressurized unit coupled to the controller and comprising a second pressurized gas, the second pressurized unit operable to selectively provide at least some of the second pressurized gas to the airways of the patient.

17. The apparatus according to claim 16 further comprising at least one valve operable to selectively couple the first pressurized gas or the second pressurized gas to the airways of the patient.

18. The apparatus according to claim 16 wherein the first pressurized gas is a positively pressurized gas and the second pressurized gas is a negatively pressurized gas.

19. A method for providing ventilation and perfusion in an airway of a patient, comprising:
selectively coupling a unit comprising a first pressure to the airways of the patient; and
selectively coupling the unit comprising a second pressure to the airways of the patient.

20. The method of claim 19 wherein the unit comprising the first pressure is different from the unit comprising the second pressure.

21. Method according to claim 19, wherein the act of selectively coupling the unit comprising a first pressure to the airways of the patient comprises moving a piston in a cylinder, the cylinder coupled to the airways of the patient, the movement of the piston causing a negative pressure in the airways of the patient.

22. Method according to claim 19, wherein the act of selectively coupling comprises open and closing at least one valve coupling the airways of the patient to the unit.

23. Method according to claim 19 further comprising controlling a gas reservoir valve connected to a gas reservoir.

24. Method according to claim 19 further comprising measuring at least one of oxygen saturation, end tidal CO₂, skin color, blood pressure, and blood flow.
